(19) United States
(12) Patent Application Publication
(10) Pub. No.: US 2004/0198793 A1
(43) Pub. Date: Oct. 7, 2004

(54) Chemical process and new intermediates

(76) Inventors: Sandor Bokotey, Esztergon (HU); Gezane Galambos, Budapest (HU); Felix Hadju, Budapest (HU); Istvan Hermecz, Budapest (HU); Agnes Horvath, Budapest (HU); Maria Uzsoki, Kistarcsa (HU); Gyulane Kiss, Budapest (HU); Lajos Nagy, Szentendre (HU); Benjamin Podanyi, Dunakeszi (HU); Attila Simon, Budapest (HU); Judit Sipos, Budapest (HU); Agota Esek Smelkone, Budapest (HU); Anna Szabo, Budapest (HU); Arpadne Vasvari, Budapest (HU)

Correspondence Address: SANOFI-SYNTHELABO INC., 9 GREAT VALLEY PARKWAY, P.O. BOX 3026, MALVERN, PA, 19355, US

(21) Appl. No.: 10/432,494

(22) PCT Filed: Nov. 27, 2001
(86) PCT No.: PCT/HU01/00121

(30) Foreign Application Priority Data
Nov. 28, 2000 (HU) P 0004741

(51) Int. Cl. C07D 403/02; A61K 31/4178

(52) U.S. Cl. 514/397; 548/312.1

(57) ABSTRACT

Process for the preparation of a compound of the general formula (I) and pharmaceutically acceptable salts and solvates thereof, (I) characterised by reacting an N-(amino-tioxo-methyl)-1H-indole-2-carboxamide of the general formula (II), with an α-halogen-ketone of the general formula (III), wherein X stands for halogen.

The subject of the invention is a new process for the preparation of a compound of the general formula (I) and pharmaceutically acceptable salts and solvates thereof

wherein

R¹ stands for hydrogen or methyl group,

R², R³, R⁴, R⁵ stand independently from each other for hydrogen, methyl, ethyl, hydroxyl, acetyloxy, methoxy, ethoxy, methyltio, trifluoromethyl or amino group or halogen atom,

R stands for hydrogen, a —(CH₂)ₙR⁶ group or a group of the general formula a.),

wherein

R⁶ stands for carboxyl or a —COOR⁷ group,

R⁷ stands for a C₁₋₄ alkyl group,

n=1, 2, 3, 4 or 5,

m=zero or 1,

R⁸ stands for a substituted phenyl group of the general formula b.),

wherein

R¹⁰ stands for hydrogen or methoxy group,

R¹¹ stands for hydrogen, methyl, ethyl, isopropyl, methoxy or ethoxy group or halogen atom,

R¹² stands for hydrogen, methyl, ethyl or methoxy group or halogen atom, or R¹¹ and R¹² form together a methylenedioxy group,

R⁹ stands for a —CH₂—R¹³, —(CH₂)₂—R¹³, —S—CH₂—R¹³, —CH₂—S—R¹³ or C₅₋₈ alkyl group,

wherein

R¹³ stands for C₅₋₇ cycloalkyl group, with the proviso that R¹⁰, R¹¹ and R¹² can not stand at the same time for hydrogen.

The compounds of the general formula (I) are cholecystokinin A (CCK-A) agonists, which are useful in the treatment of the disorders of the a gastrointestinal tract and of the central nervous system.

Preparation of the compounds of the general formula (I) is described in the publication WO 99/15525. According to the process given in the publication, the compounds of the general formula (I) are prepared by reacting the 2-aminothiazole derivatives of the general formula (IV), wherein the meaning of R⁸ and R⁹ is the same as defined above, with the acids of the general formula (V), wherein the meaning of R¹, R², R³, R⁴ and R⁵ is the same as above.

The subject of our invention is a new process for the preparation of a compound of the general formula (I) and pharmaceutically acceptable salts and solvates thereof, wherein the meanings of R, R¹, R², R³, R⁴, R⁵, R⁸ and R⁹ are as defined above, characterised by reacting an N-(amino-thioxo-methyl)-1H-indole-2-carboxamide of the general formula (II), wherein R¹, R², R³, R⁴, R⁵ and R are as defined above, with an α-halogen-ketone of the general formula (III), wherein X stands for halogen atom, R⁸ and R⁹ are as defined above, and transforming the compound of the general formula (I) or its solvate thus obtained into its salt or liberating it from its salt.

Reaction of the compounds of the general formula (II) and (III) is preferably performed in the presence of a solvent, at a temperature between room temperature and 120° C. , preferably at a temperature between 80° C. and 120° C.

As for solvent, preferably a dipolar aprotic solvent, as for instance N,N-dimethylformamide or N-methyl-2-pyrrolidone can be applied.

The resulting compound of the general formula (I) precipitates from the reaction mixture on adding it to a protic solvent, favourably to water or alcohol, or to the mixture of the two, or by the addition of ethanolamine and ethanol to the reaction mixture. The product can be isolated from the reaction mixture by filtration.

The starting N-(aminothioxomethyl)-1H-indole-2-carboxamides of the general formula (II), wherein R¹, R², R³, R⁴, R⁵ and R are the same as defined above, are new compounds.

In accordance with the above, the invention also relates to the new compounds of the general formula (II), wherein R¹, R², R³, R⁴, R⁵ and R are the same as defined above, and to the process for the preparation thereof.

The N-(aminothioxomethyl)-1H-indole-2-carboxamides of the general formula (II) of the present invention can be prepared by transforming an 1H-indole-2-carboxylic acid of the general formula (V), wherein R¹, R², R³, R⁴, R⁵ and R are the same as defined above, into an 1H-indole-2-carboxylic acid halogenide of the general formula (VI), wherein R¹, R², R³, R⁴, R⁵ and R are the same as defined above and the meaning of Hlg is halogen, reacting the resulting compound of the general formula (VI) with potassium thiocyanate, and reacting the thus obtained isothiocyanate of the general formula (VIII), wherein R¹, R², R³, R⁴, R⁵ and R are the same as defined above, with ammonia or ammonium hydroxide.

The acid halogenides of the general formula (VI), preferably the acid chlorides, can be obtained from the appropriate acids by methods known from the literature, in the case of an acid chloride for instance, favourably by refluxing with thionyl chloride, without solvent, or in the presence of an aprotic solvent.

Acylation of the potassium thiocyanate with the acid halogenide of the general formula (VI) on the effect of reflux in a dipolar-aprotic solvent, preferably in acetone or methyl ethyl ketone, takes place at the sulphur atom and results the thiocyanate derivative of the general formula (VII), which by a fast thermic rearrangement transforms into the isothiocyanate of the general formula (VIII). This compound is rather unstable, therefore it is taken into the next reaction step without isolation.

Addition of the ammonia is effected at 0-30° C., by saturation of the reaction mixture with ammonia gas or with ammonium hydroxide. The product can be isolated from the reaction mixture by filtration.

The compounds of formula (V), wherein R, R¹, R², R³, R⁴ and R⁵ are the same as defined above can be purchased, or prepared as described in the publication WO 99/15525.

The starting α-halogen-ketone derivatives of the general formula (III), wherein X stands for halogen atom, preferably bromo atom, R⁸ and R⁹ are the same as defined above, can be prepared by halogenation of the appropriate ketone derivative of the general formula (IX), wherein R⁸ and R⁹ are the same as defined above, by using methods known from the literature, preferably by reacting it with bromine, in dichloromethane.

The ketone derivative of the general formula (IX) wherein R⁸ and R⁹ are the same as defined above, can be obtained by methods known from the litarature, by Friedel-Crafts acylation of the appropriately substituted methoxybenzene with the appropriate acid chloride, in the presence of Lewis acids, e.g. TiCl₄, AlCl₃ or FeCl₃, in aprotic solvents, favourably in dichloromethane (publication WO 99/15525).

The acid chlorides where R⁹ means an alkyl group —CH₂—R¹³, (CH₂)₂—R¹³, or (C₅₋₈) can be prepared from the appropriate acids available on the market, by general methods known in the literature, as for instance by reaction with thionyl chloride, oxalyl chloride or with POCl₃/DMF .

Compounds of the general formula (III) wherein R⁹ stands for cycloalkylmethylthio- or cyckloalkylthiomethyl-group and the meaning of R⁸ is the same as defined above, are new compounds.

Our invention relates furthermore to the new compounds of the general formula (III) wherein R⁹ stands for cycloalkylmethylthio- or cyckloalkylthiomethyl-group and the meaning of R⁸ is the same as defined above, and the process for the preparation thereof.

According to our invention the new compounds of the general formula (III), wherein R⁹ stands for cycloalkylmethylthio- or cyckloalkylthiomethyl-group and the meaning of R⁸ is the same as defined above, can be prepared by acylation of a methoxybenzene of the general formula (X), wherein the meaning of R¹⁰, R¹¹ and R¹² is the same as defined above, with an acid chloride of the general formula (XI), wherein R¹⁴ stands for (C₅₋₇) cycloalkyl-group, o means 1 or 2 and p means zero or 1, in the presence of an aprotic solvent, preferably dichloromethane, and a Lewis acid, preferably titane tetrachloride or aluminium chloride, at 0-5° C., followed by halogenation in an aprotic solvent by methods used in the literature, favourably by bromination in dichloromethane with bromine.

The acid chloride of the general formula (XI), wherein the meaning of R¹⁴, o and p is the same as defined above, can be obtained from an acid of the general formula (XII), wherein the meaning of R¹⁴, o and p is the same as defined above, by reaction with thionyl chloride or oxalyl chloride in an aprotic solvent, preferably in dichloromethane.

The acid of the general formula (XII), wherein the meaning of R¹⁴, o and p is the same as defined above can be prepared by alkylating an appropriate cycloalkylmethylthio, or cycloalkylthio compound of the general formula (XIII), in alkaline media, with an bromoalkylcarboxylic acid esters of the general formula (XIV), wherein R¹⁵ stands for methyl or ethyl group and the meaning of o is the same as defined above, preferably with bromoacetic acid methyl ester (o=1), or with 3-bromopropionic acid ethyl ester (o=2), and by subsequent hydrolysis of the ester group.

The cycloalkylthio compound of the general formula (XIII) can be purchased, or it can be prepared from the appropriate cycloalkylmethyl bromide of the general formula (XV), available on the market, by reacting it in a protic solvent, favourably in ethanol with thiocarbamide and by hydrolysing the resulting S-alkyl-isothiuronium salt of the general formula (XVI), wherein the meaning of R¹⁴ is the same as defined above, and p=zero, with an alkali hydroxide solution, preferably sodium hydroxide solution.

Further details of the invention are demonstrated by the following examples, without limiting the claims to the examples.

### EXAMPLES

### Preparation of Compounds of the General Formula (II)

### 1.) Preparation of the N-(aminothioxomethyl)-5,7-dimethyl-1-(methoxycarbonylmethyl)-1H-indole-2-carboxamide

The mixture of 78.35 g (0.30 mol) of 5,7-dimethyl-2-(hydroxycarbonyl)-1H-indole-1-acetic acid methyl ester, 390 ml of dichloromethane and 24.08 ml, (39.26 g=0.33 mol) of thionyl chloride is heated under reflux conditions and stirring. The solid starting material dissolves in about 45 minutes. The dark solution is heated under reflux for an additional hour, then it is evaporated in vacuum and 150 ml of toluene is distilled through the residue. The solid residue is dissolved in 500 ml of methyl ethyl ketone at 40-50° C. and added in about 20 minutes to the stirred refluxing suspension of 29.15 g (0.30 mol) of potassium thiocyanate and 150 ml of methyl ethyl ketone. The mixture is refluxed for an additional 30 minutes, then it is cooled to +5° C. Under stirring and external cooling ammonia gas is introduced into the mixture, while its temperature elevates to 34° C. and the product precipitates from the solution in the form of yellow crystals.

When the temperature starts to decrease (after about 10 minutes) the gas inlet is stopped and the reaction mixture is allowed to stand for another hour in a bath of 5-10° C., then 650 ml of water is added to the mixture under stirring and the resulting two-phase suspension is stirred for 25 minutes. The crystals are filtered off in vacuum, washed subsequently with water and acetone. 87.03 g of pale-yellow crystals of the crude product are obtained, mp: 226-231° C.

### 2.) Preparation of N-(aminothioxomethyl)-5-methyl-1-(methoxycarbonylmethyl)-1H-indole-2-carboxamide

The mixture of 4.46 g (18 mmol) of 5-methyl-2-(hydroxycarbonyl)-1H-indole-1-acetic acid methyl ester, 22 ml of dichloromethane, 1.5 ml (2.4 g=20 mmol) of thionyl chloride and 3 drops of N,N-dimethylformamide is heated under reflux conditions for 2 hours. The thick suspension turns into a yellow solution, which is evaporated in vacuum and 10 ml of toluene is distilled through the residue. The thus obtained acid chloride is dissolved in 50 ml of acetone and dropped to the boiling suspension of 1.75 g (18 mmol) of potassium thiocyanate and 10 ml of acetone. The reaction mixture is boiled for 45 minutes, then it is cooled from ice-water bath to 4-6° C. and ammonia gas is introduced as long as the exothermic reaction lasts. The mixture is then stirred on ice-water bath for 1 hour, 60 ml of water is added to it, and stirring is continued for another hour. The crystals are filtered off in vacuum, washed with water to obtain 4.45 g of the title compound in the form of beige-coloured crystals, mp: 222-224° C.

### 3.) Preparation of N-(aminothioxomethyl)-3,5-dimethyl-1-(methoxycarbonylmethyl)-1H-indole-2-carboxamide

The mixture of 2.2 g (8.48 mmol) of 3,5-dimethyl-2-(hydroxycarbonyl)-1H-indole-1-acetic acid methyl ester, 12 ml of dichloromethane, 1.2 g (11 mmol)˜0.75 ml of thionyl chloride and 2 drops of N,N-dimethylformamide is heated under reflux conditions for 1.5 hours, while the thick suspension turns into a yellow solution, then it is evaporated in vacuum and 5 ml of toluene is distilled through the residue. A suspension made of 0.85 g (8.4 mmol) of potassium thiocyanate and 5 ml of acetone is heated to 50° C. and to it the acid chloride dissolved in 50 ml of acetone is dropped. The reaction mixture is boiled for 30 minutes, then it is cooled from ice-water bath to 6-8° C. and ammonia gas is introduced until the temperature of the reaction mixture is increasing. The mixture is then stirred on ice-water bath for 1 hour, 25 ml of water is added to it, and stirring is continued for another hour. The crystals are filtered off in vacuum, washed with water-acetone 1:1 mixture to obtain 1.75 g of the title compound in the form of beige-coloured crystals, mp: 210-212° C.

### 4.) Preparation of the mixture of N-(aminothioxomethyl)-4,5-dimethyl-1-(methoxycarbonylmethyl)-1H-indole-2-carboxamide and N-(aminothioxomethyl)-5,6-dimethyl-1-(methoxycarbonylmethyl)-1H-indole-2-carboxamide

5.23 g (20 mmol) of the mixture of 4,5-dimethyl-2-(hydroxycarbonyl)-1H-indole-1-acetic acid methyl ester and 5,6-dimethyl-2-(hydroxycarbonyl)-1H-indole-1-acetic acid methyl ester is refluxed for 2 hours in 25 ml of dichloromethane, in the presence of 2.6 g (22 mmol)˜1.6 ml of thionyl chloride and 3 drops of N,N-dimethylformamide, while the thick suspension turns into a yellow solution, then it is evaporated in vacuum and 10 ml of toluene is distilled through the residue. A suspension made of 1.98 g (20 mmol) of potassium thiocyanate and 10 ml of acetone is heated to 50° C. and the acid chloride dissolved in 40 ml of acetone is dropped to it. The reaction mixture is boiled for 45 minutes, then it is cooled from ice-water bath to 0-5° C. and ammonia gas is introduced until the temperature of the reaction mixture is increasing. The mixture is then stirred on ice-water bath for 1 hour, 60 ml of water is added to it, and stirring is continued for another hour. The crystals are filtered off in vacuum, washed with water-acetone 1:1 mixture to obtain 5.15 g of the title compound in the form of beige-coloured crystals, mp: 214-216° C.

### 5.) Preparation of N-(aminothioxomethyl)-5-methoxy-1-(methoxycarbonyl methyl)-1H-indole-2-carboxamide

1.2 g (4.5 mmol) of 5-methoxy-2-(hydroxycarbonyl)-1H-indole-1-acetic acid methyl ester and 0.65 g (5 mmol)˜0.4 ml of thionyl chloride in 10 ml of dichloromethane, in the presence of 1drop of N,N-dimethylformamide are heated under reflux conditions for 1 hour. The dark solution is then evaporated in vacuum and 10 ml of toluene is distilled through the residue. The acid chloride is refluxed in 10 ml of acetone with 0.44 g (4.5 mmol) of potassium thiocyanate for 30 minutes, then the mixture is cooled from ice-water bath to 0-5° C. and 1 ml of 25% ammonium hydroxide is added to it. The thick suspension is diluted with 5 ml of acetone, stirred at room temperature for 30 minutes. The crystals are filtered off in vacuum, washed subsequently with water and acetone to obtain 1.16 g of yellowish-drab crystals of the title compound, mp: 202-206° C.

### 6.) Preparation of N-(aminothioxomethyl)-5,7-dimethyl-1H-indole-2-carboxamide

4.73 g (0.025 mol) of 5,7-dimethyl-1H-indole-2-carboxylic acid are suspended in 40 ml of dichloromethane, to the mixture 2.1 ml (0.028 mol) of thionyl chloride and 2 drops of N,N-dimethylformamide as catalyst are added. The suspension is refluxed for 5 hours, then evaporated. The residue is dissolved in 30 ml of acetone and added to the refluxing solution of 2.43 g (0.025 mol) of potassium thiocyanate and 10 ml of acetone and heated under reflux conditions for 30 minutes. The reaction mixture is cooled to 10° C. and ammonia gas is introduced into the mixture for a period of 15 minutes. The precipitated solid material is filtered off, the filtrate solution is poured onto 100 ml of water, the precipitated product is washed with ethanol. Product: 3.78 g pale-yellow crystals of the title compound, mp: 220-223° C.

### 7.) Preparation of N-(aminothioxomethyl)-5,7-dimethyl-1-(4-methoxycarbonylbenzyl)-1H-indole-2-carboxamide

3.59 g (10.7 mmol) of 5,7-dimethyl-1-(4-methoxycarbonylbenzyl)-1H-indole-2-carboxylic acid and 1.26 g (11.2 mmol)˜0.8 ml of thionyl chloride are refluxed for 1 hour in 20 ml of dichloromethane, in the presence of 1 drop of N,N-dimethylformamide. The dark solution is then evaporated in vacuum and 10 ml of toluene is distilled through the residue. The acid chloride is refluxed in 10 ml of acetone with 1.05 g (10.7 mmol) of potassium thiocyanate for 30 minutes, then the mixture is cooled from ice-water bath to 0-5° C. and 2 ml of 25% ammonium hydroxide is added to it. The thick suspension is diluted with 10 ml of acetone and 10 ml of acetonitrile, stirred at room temperature for 30 minutes, then under stirring 90 ml of water is added to the suspension and stirring is continued for another 30 minutes. The crystals are filtered off in vacuum, washed with water to obtain 3.17 g of butter-coloured crystals of the title compound, mp: 222-224° C.

### 8.) Preparation of N-(aminothioxomethyl)-5,7-dimethyl-1-(3-methoxycarbonylbenzyl)-1-H-indole-2-carboxamide

1.8 g (5.3 mmol) of 5,7-dimethyl-1-(3-methoxycarbonylbenzyl)-1H-indole-2-carboxylic acid and 0.65 g (5.6 mmol)˜0.4 ml of thionyl chloride are refluxed for 1 hour in 10 ml of dichloromethane, in the presence of 1 drop of N,N-dimethylformamide. The dark solution is then evaporated in vacuum and 10 ml of toluene is distilled through the residue. The acid chloride is refluxed in 25 ml of acetone with 0.53 g (5.3 mmol) of potassium thiocyanate for 30 minutes, then the mixture is cooled from ice-water bath to 5° C. and 1 ml of 25% ammonium hydroxide is added to it. The thick suspension is stirred at room temperature for 60 minutes, diluted with 50 ml of water and stirred for another 30 minutes. The crystals are filtered off in vacuum, washed with water to obtain 1.9 g of butter-coloured crystals of the title compound, mp: 198-199° C.

### 9.) Preparation of N-(aminothioxomethyl)-1-(ethoxycarbonylethyl)-1H-indole-2-carboxamide

1.5 g (5.7 mmol) of 2-(hydroxycarbonyl)-1H-indole-1-propionic acid ethyl ester and 0.71 g (6 mmol)˜0.45 ml of thionyl chloride are refluxed for 40 minutes in 12 ml of dichloromethane, in the presence of 1 drop of N,N-dimethylformamide. The dark solution is then evaporated in vacuum and 10 ml of toluene is distilled through the residue. The acid chloride is refluxed in 15 ml of acetone with 0.56 g (5.7 mmol) of potassium thiocyanate for 1 hour, then the mixture is cooled from ice-water bath to 0-5° C. and 1.25 ml of 25% ammonium hydroxide is added to it. The suspension is stirred at room temperature for 30 minutes, diluted with 15 ml of water and stirred for another 30 minutes. The crystals are filtered off in vacuum, washed with water to obtain 1.16 g of yellow crystals of the title compound, mp: 160-162° C.

### 10.) Preparation of the mixture of N-(aminothioxomethyl)-4,5-dimethyl-1-(3-methoxycarbonylbenzyl)-1H-indole-2-carboxamide and N-(aminothioxomethyl)-5,6-dimethyl-1-(3-methoxycarbonylmethyl)-1H-indole-2-carboxamide

From the mixture of 4,5-dimethyl-1-(3-methoxycarbonylbenzyl)-1H-indole-2-carboxylic acid and 5,6-dimethyl-1-(3-methoxycarbonylmethyl)-1H-indole-2-carboxylic acid according to Example 9. butter-coloured crystals of the title compound mixture are obtained, mp: 193-196° C.

### Preparation of the Compounds of the General Formula (III)

### 11.) Preparation of 2-bromo-1-(2,4-dimethoxyphenyl)-3-cyclohexyl-1-propanone

### A) Preparation of 3-cyclohexylpropionyl chloride

23.43 g (0.15 mol) of 3-cyclohexylpropionic acid are dissolved in 150 ml of dichoromethane, to the solution 14.6 ml (0.2 mol) of thionyl chloride and 3 drops of dimethylformamide are added and the mixture is refluxed for 3 hours. The reaction mixture is evaporated to obtain 25.72 g of the title product, which can be used without further purification for the next step.

### B) Preparation of 1-(2,4-dimethoxyphenyl)-3-cyclohexyl-1-propanone

The suspension made of 6.8 g (0.042 mol) of iron(III)chloride and 30 ml of dichloromethane is cooled to 5° C. and to it is added dropwise, and under cooling, the mixture of 5.53 g (0.04 mol) of 1,3-dimethoxy-benzene and 7.68 g (0.044 mol) of 3-cyclohexylpropionyl chloride. The reaction mixture is stirred at room temperature for 3 hours, then it is poured to 100 ml of ice-water, the resulting emulsion is stirred for 30 minutes, the phases are separated and the organic phase is stirred with 40 ml of 1N sodium hydroxide solution for 30 minutes. The organic layer is washed with saturated sodium chloride solution and evaporated. The residue is crystallised from the 10/1 mixture of petroleum ether/methanol to obtain 8.0 g white crystals of the title compound, mp: 34-35° C.

### C) Preparation of 2-Bromo-1-(2,4-dimethoxyphenyl)-3-cyclohexyl-1-propanone

To the solution of 2.62 g (9.48 mmol) of 1-(2,4-dimethoxyphenyl)-3-cyclohexyl-1-propanone in 10 ml of dichoromethane is dropped at room temperature the solution of 1.6 g (10 mmol) of bromine in 5 ml of dichoromethane. The reaction mixture is stirred at room temperature for 2 hours, then 15 ml of water is added and stirring is continued for another 30 minutes. The phases are separated, the organic layer is washed with 15 ml of water and evaporated. The residue is taken up in a small amount of ethanol, the precipitated crystals are filtered off in vacuum. 1.54 g white crystals of the title compound are obtained, mp: 83-89° C.

### 12.) Preparation of 2-bromo-1-(2,5-dimethoxyphenyl)-3-cyclohexyl-1-propanone

The proccess described in Example 11. is followed, starting from 1,4-dimethoxybenzene instead of 1,3-dimethoxybenzene. The product obtained by evaporation is an oil, weight: 9.8 g.

### 13.) Preparation of 2-bromo-1-(2,5-dimethoxy-4-methylphenyl)-3cyclohexyl-1-propanone

### A) Preparation of the 4-cyclohexylbutyryl chloride

25.0 g (0.147 mol) of 4-cyclohexylbutyric acid are dissolved in 50 ml of dichoromethane, to the solution 16 ml (0.22 mol) of thionyl chloride and 3 drops of dimethylformamide are added and the mixture is refluxed for 2 hours. The product obtained by evaporation is used without further purification for the next step.

### B) Preparation of 1-(2,5-dimethoxy-4-methylphenyl)-4-cyclohexyl-1-cyclohexyl-1-butanone

The solution of 4.56 g (0.03 mol) of 2,5-dimethoxytoluene and 6.23 g (0.033 mol) of cyclohexylbutyryl chloride in 30 ml of dichloromethane is cooled to 5° C. and to it is added dropwise 5.69 g (0.03 mol) of titanium(FV) chloride. The reaction mixture is allowed to warm up to room temperature, stirred for another 2 hours, poured onto the mixture of 70 ml of ice-water and 30 ml of conc. hydrochloric acid and stirred for 30 minutes. The phases are separated, the aqueous phase is extracted with 30 ml of dichloromethane, the united organic phase is stirred for 30 minutes with 40 ml of 1N sodium hydroxide solution, then with 40 ml of saturated sodium chloride solution, dried and evaporated. The residue is taken up in 15 ml of methanol, the crystals are filtered off in vacuum. Product: 6.65 g white crystals of the title compound, mp: 52-53° C.

### C) Preparation of 2-bromo-1-(2,5-dimethoxy-4-methylphenyl)-4-cyclohexyl-1-butanone

To the solution of 3.04 g (0.01 mol) of 1-(2,5-dimethoxy-4-methylphenyl)-4-cyclohexyl-1-butanone in 20 ml of dichoromethane is dropped at room temperature the solution of 1.6 g (10 mmol) of bromine in 5 ml of dichoromethane. The reaction mixture is stirred at room temperature for 2 hours, then with 25 ml of water for 15 minutes. The phases are separated, the organic layer is washed with 15 ml of water and evaporated. The residue is taken up in a small amount of ethanol, the precipitated crystals are filtered off in vacuum. 2.64 g yellow crystals of the title compound are obtained, mp: 75-77° C.

### 14.) Preparation of 2-bromo-1-(2,5-dimethoxy-4-chlorophenyl)-4-cyclohexyl-1-butanone

### A) Preparation of 4-cyclohexylbutyryl chloride

125 ml (1.625 mol) of dimethylformamide is cooled to −5° C. and 201 g (1.31 mol) of phosphoryl chloride is added to it, while keeping the temperature at 0-5° C. The reaction mixture is allowed to warm up to 15° C. and 204.3 g (1.25 mol) of 4-cyclohexylbutyric acid is added. The mixture is stirred at room temperature for 4 hours, then the phases are separated. The upper layer, 220.8 g of thick viscosine solution, is taken into the next step without further purification.

### B) Preparation of 1-(2,5-dimethoxy-4 chlorophenyl)-4-cyclohexyl-1-butanone

Into 480 ml of dichloromethane at 5° C. 175.6 g (1.05 mol) of iron(III) chloride are added, then at that temperature 172.61 g (1 mol) of 2,5-dimethoxychlorobenzene and the 4-cyclohexybutyryl chloride are added. The reaction mixture is stirred for 4 hours, while keeping the temperature below 35° C., then it is poured to 1500 ml of ice-water and the phases are separated. The aqueous layer is extracted with dichloromethane, the united organic phase is washed 1N sodium hydroxide solution, then with saturated sodium chloride solution and evaporated. The residue is suspended in methanol, the crystalline product is filtered off in vacuum. 289.5 g pale-yellow crystals of the title compound are obtained, mp: 79-80° C.

### C) 2-Bromo-1-(2,5-dimethoxy-4-chlorophenyl)-4-cyclohexyl-1-butanone

243.3 g (0.749 mol) of 1-(2,5-dimethoxy-4-chlorophenyl)-4-cyclohexyl-1-butanone are dissolved in 980 ml of dichoromethane. Into the pale-yellow solution 119.7 g (0.749 mol) of bromine in 380 ml of dichoromethane are dropped at room temperature, in a period of 40 minutes. The brown, clear reaction mixture is stirred at room temperature for 30 minutes, then 1500 ml of water is added and the mixture is stirred for 30 minutes The separated organic layer is stirred again with 750 ml of water for 30 minutes. The phases are separated, the organic layer is evaporated. The oily residue is taken up in 450 ml of ethanol, the precipitated crystals are filtered off in vacuum. 288.4 g light yellow crystals of the title compound are obtained, mp: 73-74° C.

### 15.) Preparation of 2-bromo-1-(2,5-dimethoxy-4-chlorophenyl)-1-decanone

### A) Preparation of 1-(2,5-dimethoxy-4-chlorophenyl)-1-decanone

The solution of 8.63 g (0.05 mol) of 2,5-dimethoxychlorobenzene and 10.49 g (0.055 mol) of decanoyl chloride in 50 ml of dichloromethane is cooled to 5° C. and to it is added dropwise 9.48 g (0.05 mol) of titanium(IV) chloride. The reaction mixture is allowed to warm up to room temperature, stirred for another 2 hours, poured onto the mixture of 100 ml of ice-water and 40 ml of conc. hydrochloric acid and stirred for 30 minutes. The phases are separated, the aqueous phase is extracted with 30 ml of dichloromethane, the united organic phase is stirred for 30 minutes with 60 ml of 1N sodium hydroxide solution then with 60 ml of saturated sodium chloride solution, dried and evaporated. The residue is taken up in 40 ml of methanol, the crystals are filtered off in vacuum. Product: 12.15 g white crystals, mp: 53-54° C.

### B) Preparation of 2-bromo-1-(2,5-dimethoxy-4-chlorophenyl)-1-decanone

To the solution of 3.27 g (0.01 mol) of 1-(2,5-dimethoxy-4-chlorophenyl)-1-decanone in 20 ml of dichoromethane the solution of 1.6 g (10 mmol) of bromine in 11 ml of dichoromethane is dropped at room temperature. The reaction mixture is stirred at room temperature for 30 minutes, then with 30 ml of water for 30 minutes. The phases are separated, the organic layer is washed with 30 ml of water and evaporated to obtain 3.78 g of the title compound in the form of an oil.

### 16.) Preparation of 2-bromo-1-(5-bromo-2,5-dimethoxyphenyl)-4-cyclohexyl-1-butanone

### A) Preparation of 1-(5-bromo-2,5-dimethoxyphenyl)-4-cyclohexyl-1-butanone

The solution of 8.68 g (0.04 mol) of 1-bromo-2,4-dimethoxybenzene and 7.55 g (0.04 mol) of cyclohexylbutyric acid in 50 ml of dichloromethane is cooled to 0° C. and to it is added dropwise 4.4 ml (0.04 mol) of titanium (V) chloride. The reaction mixture is allowed to warm up to room temperature, stirred for another 5 hours. The reaction mixture is worked-up as described in 13. B) to obtain 10.05 g white crystals of the title compound, mp: 82-87° C.

### B) Preparation of 2-bromo-1-(5-bromo-2,5-dimethoxyphenyl)-4-cyclohexyl-1-butanone

To the solution of 3.69 g (0.01 mol) of 1-(5-bromo-2,5-dimethoxyphenyl)-4-cyclohexyl-1-butanone in 20 ml of dichoromethane the solution of 1.6 g (10 mmol) of bromine in 15 ml of dichoromethane is dropped at room temperature. The reaction mixture is stirred at room temperature for 30 minutes, then with 50 ml of water for 30 minutes. The phases are separated, the organic layer is washed with 2×30 ml of water and evaporated. The residue is taken up in a small amount of cold ethanol, the precipitated crystals are filtered off in vacuum to obtain 3.09 g white crystals of the title compound, mp: 96-104° C.

### 17.) Preparation of 1-(2,5-dimethoxy-4-chlorophenyl)-2-cyclohexylmethylthio-1-ethanone

### A) Preparation of cyclohexylmethylthioacetic acid

The mixture of 8.85 g (0.05 mol) of cyclohexylmethyl bromide, 100 ml of ethanol and 3.8 g (0.05 mol) of thiocarbamide is heated under reflux conditions for 12 hours, cooled to room temperature, 10 g (0.25 mol) of sodium hydroxide dissolved in 50 ml of water is added and the mixture is refluxed for 2 hours. After cooling to room temperature 7.65 g (0.05 mol) of bromoacetic acid methyl ester in 20 ml of ethanol is added and reflux is continued for 3 hours. Then, under stirring and external cooling the solution of 7 ml of conc. sulfuric acid and 80 ml of water is dropped to the reaction mixture (pH=1), the oily product is extracted with 2×150 ml of ethyl acetate, the organic phase is washed with 200 ml of water, dried over sodium sulfate and evaporated. The residual 9.1 g raw product is purified by coloumn chromatography using toluene-methanol 10:1 mixture eluent. Collecting and evaporating the pure fractions the title compound was obtained in the form of an oil, which was used for the next step without further purification.

### B) Preparation of cyclohexylmethylthioacetyl chloride

To the solution made of 5.63 g (0.03 mol) of cyclohexylmethylthioacetic acid, and 3.3 ml (0.045 mol) of thionyl chloride in 20 ml of dichoromethane 3 drops of dimethylformamide are added and the mixture is refluxed for 2 hours. The product obtained after evaporation is used without further purification for the next step.

### C) Preparation of 1-(2,5-dimethoxy-4-chlorophenyl)-2-cyclohexylmethylthio-1-ethanone

The solution of 4.70 g (0.027 mol) of 2,5-dimethoxy-4-chlorobenzene and 6.20 g (0.03 mol) of cyclohexylmethylthioacetyl chloride in 30 ml of dichloromethane is cooled to 5° C. and to it is added dropwise 2.96 ml (0.027 mol) of titanium(IV) chloride. The reaction mixture is allowed to warm up to room temperature, stirred for another 2 hours, poured onto the mixture of 80 ml of ice-water and 20 ml of conc. hydrochloric acid, stirred for 30 minutes. The phases are separated, the aqueous phase is extracted with 30 ml of dichloromethane, the united organic phase is stirred for 30 minutes with 40 ml of 1N sodium hidroxide solution, the organic phase is washed with 40 ml of saturated sodium chloride solution, dried and evaporated. The residue is taken up in 20 ml of methanol. The resulting crystals are filtered off in vacuum, to obtain 5.33 g white crystals of the title compound, mp: 51-53° C.

### D) Preparation of 2-bromo-1-(2,5-dimethoxy-4-cholorophenyl)-2-cyclohexylmethylthio-1-ethanone

To 1.85 g (0.0055 mol) of 1-(2,5-dimethoxy-4-chlorophenyl)-2-cyclohexylmethylthio-1-ethanone dissolved in 30 ml of dichloromethane is added at 10° C., in a period of 10 minutes, under stirring the solution of 0.87 g (0.0055 mol) of bromine in 30 ml dichloromethane. The mixture is stirred at room temperature for 15 minutes, washed twice with 40 ml of water and evaporated to obtain 2.1 g of the title compound as a thick oil.

### 18.) Preparation of 1-(2,5-dimethoxy-4-chlorophenyl)-3-cyclohexylthio-1-propanone

### A) Preparation of 3-cyclohexylthio-1-propionic acid

To the mixture of 11.6 g (0.1 mol) of cyclohexylmercaptan, 70 ml of ethanol and 12 g (0.3 mol) of sodium hydroxide dissolved in 50 ml of water is added at 10-15° C., under external heating, the solution of 18.1 g (0.1 mol) of 3-bromopropionic acid ethyl ester in 100 ml of ethanol. The reaction mixture is refluxed for 5 hours, cooled to room temperature and under external heating and stirring the solution of 15 ml of conc. sulfuric acid in 100 ml of water is added to it. The reaction mixture is extracted twice with 200 ml of ethyl acetate. The organic layer is washed with 200 ml of water, dried over anhydrous sodium sulfate and evaporated. The residual 16.5 g of crude product is purified by chromatography using 10:1 toluene-methanol mixture as eluent. Collecting and evaporating the pure fractions the title compound was obtained in the form of a pale oil, which was used for the next step without further purification.

### B) Preparation of 3-cyclohexylthio-1-propionyl chloride

To the solution made of 6.99 g (0.037 mol) of cyclohexylmethylthiopropionic acid, 4.1 ml (0.056 mol) of thionyl chloride and of 30 ml of dichloromethane 3 drops of dimethylformamide are added and the mixture is refluxed for 1 hours. The product obtained after evaporation is used without further purification for the next step.

### C) Preparation of 1-(2,5-dimethoxy-4-chlorophenyl)-3-cyclohexylthio-1-propanone

The solution of 5.18 g (0.03 mol) of 2,5-dimethoxy-4-chlorobenzene and 6.20 g (0.03 mol) of cyclohexylthiopropionyl chloride in 30 ml of dichloromethane is cooled to 5° C. and to it is added dropwise 3.3 ml (0.03 mol) of titanium (IV) chloride. The reaction mixture is stirred at 5-10° C. for 3 hours, then poured onto the mixture of 80 ml of ice-water and 20 ml of conc. hydrochloric acid, stirred for 30 minutes. The phases are separated, the aqueous phase is extracted with 30 ml of dichloromethane, the united organic phase is stirred for 30 minutes with 40 ml of 1N sodium hydroxide solution, the organic phase is washed with 40 ml of saturated sodium chloride solution, dried and evaporated. The residue is taken up in 100 ml of petroleum ether to obtain 4.12 g oily product of the title compound.

### D) Preparation of 2-bromo-1-(2,5-dimethoxy-4-chlorophenyl)-3-cyclohexylthio-1-propanone

To 0.8 g (0.002 mol) of 1-(2,5-dimethoxy-4-chlorophenyl)-3-cyclohexylthio-1-propanone dissolved in 30 ml of dichloromethane is added at 10° C., in a period of 10 minutes, under stirring the solution of 0.32 g (0.002 mol) of bromine in 20 ml dichloromethane. The mixture is stirred at room temperature for 15 minutes, washed twice with 40 ml of water and evaporated to obtain 0.95 g of the title compound as a thick oil.

### Preparation of the Compounds of the General Formula (I)

### 19.) Preparation of 2,-[[[4-(2,5-dimethoxy-4-chlorophenyl)-5-cyclohexylethylthiazol-2-yl]-amino]-carbonyl]-5,7-dimethyl-1H-indole-1-acetic acid methyl ester

To 880 ml of N,N-dimethylformamide at room temperature, under stirring are added 195.72 g (0.613 mol) of N-(aminothioxomethyl)-5,7-dimethyl-1-(methoxycarbonylmethyl)-1H-indole-2-carboxamide and 247.42 g (0.613 mol) of 2-bromo-1-(2,5-dimethoxy-4-cholorophenyl)-4-cyclohexyl-1-butanone. The suspension under stirring turnes at 83° C. into a dark solution which is stirred at 95-105° C. inner temperature for 3,5 hours. The solution is then cooled to 80° C., 85 ml (62 g=0.613 mol) of triethylamine is added to it, stirred for 5 minutes and 2640 ml of ethanol is added to it. The pale solution cooles to 45-55° C. and crystallisation starts. The reaction mixture is cooled to room temperature under stirring. The crystals are filtered off in vacuum, washed with ethanol to obtain 315.91 g white cystals of the crude product, mp.: 193-195° C.

### 20.) Preparation of 2-[[[4-(2,5-dimethoxyphenyl)-5-cyclohexylmethylthiazol-2-y l]-amino]-carbonyl]-5,7-dimethyl-1-H-indole-1-acetic acid methyl ester

To 15 ml of N,N-dimethylformamide at room temperature under stirring are added 1.6 g (5 mmol) of N-(aminothioxomethyl)-5,7-dimethyl-1(methoxycarbonylmethyl)-1H-indole-2-carboxamide and 2.22 g (6.26 mmol) of 2-bromo-1-(2,5-dimethoxyphenyl)-3-cyclohexyl-1-propanone. The pale-brown solution is stirred for 3 hours at 105° C. inner temperature, cooled to 80° C., diluted with 90 ml of 50% ethanol and cooled to room temperature under stirring. The resulting crystals are filtered off in vacuum, washed with 50% aqueous ethanol to obtain 2.1 g yellow crystals of the crude product, mp: 89-92° C.

### 21.) Preparation of 2-[[[4-(2,5-dimethoxy-4-methylphenyl)-5-cyclohexylethylthiazol-2-yl]-amino]-carbonyl]-5,7-dimethyl-1H-indole-1-acetic acid methyl ester

To

15 ml of N,N-dimethylformamide at room temperature under stirring are added 1.6 g (5 mmol) of N-(aminothioxomethyl)-5,7-dimethyl-1-(methoxycarbonylmethyl)-1H-indole-2-carboxamide and 1.97 g (5.2 mol) of 2-bromo-1-(2,5-dimethoxy-4-methylphenyl)-4-cyclohexyl-1-butanone. The suspension turnes at 80° C. under stirring into an orange-coloured solution which is stirred at 100-105°0 C. inner temperature for 3 hours. The reaction mixture is cooled under stirring to room temperature and poured onto 45 ml of water. The butter-coloured suspension is stirred at room temperature for 30 minutes, the crystals are filtered off in vacuum, crystallized from N,N-dimethylformamide—water mixture. 2.82 g pale beige-coloured crystals are obtained as crude product, mp: 154-162° C.

### 22.) Preparation of 2-[[[4-(2,5-dimethoxy-4-methylphenyl)-5-cyclohexylethylthiazol-2yl]-amino]-carbonyl]-5-methoxy-1-H-indole-1-acetic acid methyl ester

To 10 ml of N,N-dimethylformamide at room temperature under stirring are added 1 g (3.1 mmol) of N-(aminothioxomethyl)-5-methoxy-1-(methoxycarbonylmethyl)-1H-indole-2-carboxamide and 1.19 g (3.1 mol) of 2-bromo-1-(2,5-dimethoxy-4-methylphenyl)-4-cyclohexyl-1-butanone. The orange-coloured solution is stirred at 100-105° C. inner temperature for 4 hours, cooled to 80° C. under stirring, 0.45 ml of triethylamine is added to it, stirred for 5 minutes, diluted with 40 ml of 96% ethanol. After a few minutes crystallisation starts from the clear brown solution. The suspension is stirred for 30 minutes. Crystals are filtered off in vacuum, washed with 96% ethanol to obtain 1.28 g white crystals of the product, mp: 173-175° C.

### 23.) Preparation of 4-[[2-[[[4-(2,5-dimethoxy-4-chlorophenyl)-5-cyclohexylethylthiazol-2-yl]amino]carbonyl]-5,7-dimethyl-1-H-indole-1-yl]-methyl]benzoic acid methyl ester

To

15 ml of N,N-dimethylformamide at room temperature under stirring are added 3.0 g (7.6 mmol) of N-(aminothioxomethyl)-5,7-dimethyl-1-(4-methoxycarbonylbenzyl)-1H-indole-2-carboxamide and 3.07 g (7.6 mmol)2-bromo-1-(2,5-dimethoxy-4-chlorophenyl)-4-cyclohexyl-1-butanone. The suspension turnes at 72° C. under stirring into a brown solution which is stirred at 100-105° C. inner temperature for 3.5 hours. To the reaction mixture cooled to 65° C. 1.1 ml (0.77 g=7.6 mmol) of triethylamine is added, stirred for 5 minutes, cooled to room temperature and poured onto the ice-cold mixture of 45 ml of ethanol and 25 ml of water. The butter-coloured suspension is stirred at room temperature for 60 minutes, the crystals are filtered off in vacuum, washed with water to obtain 4.9 g pale-beige coloured crystals of the title compound, mp: 199-200° C.

### 24.) Preparation of 3-[[2-[[[4-(2,5-dimethoxy-4-chlorophenyl)-5-octylthiazol-2yl] amino] carbonyl]-5,7-dimethyl-1H-indole-1-yl]methyl]benzoic acid methy ester

To 10 ml of N,N-dimethylformamide at room temperature under stirring are added 1.78 g (4.5 mmol) of N-(aminothioxomethyl)-5,7-dimethyl-1-(3-methoxycarbonylbenzyl)1H-indole-2-carboxamide and 1.83 g (4.5 mmol) of 2-bromo-1-(2,5-dimethoxy-4-chlorophenyl)-1-decanone. The suspension turnes at 38° C. under stirring into a brown solution which is stirred at 100-105° C. inner temperature for 4 hours. To the reaction mixture cooled to 80° C. 0.6 ml (0.44 g=4.5 mmol) of triethylamine is added, stirred for 5 minutes, cooled to room temperature and poured onto 30 ml of ethanol. The butter-coloured suspension is stirred at room temperature for 60 minutes, then from ice-water bath for 30 minutes. The crystals are filtered off in vacuum, washed with ethanol to obtain 2.61 g pale-beige coloured crystals of the title compound, mp: 195-197° C.

### 25.) Preparation of 2-[[[4-(2,5-dimethoxy-4-chlorophenyl)-5-cyclohexyleyhly-thiazol-2-yl]amino]carbonyl]-5-methyl-1H-indole-1-acetic acid methyl ester

To 18 ml of N,N-dimethylformamide at room temperature under stirring are added 3.56 g (11.7 mmol) of N-(aminothioxomethyl)-5-methyl-1-(methoxycarbonylmethyl)-1H-indole-2-carboxamide and 4.75 g (11.7 mol) of 2-bromo-1-(2,5-dimethoxy-4-chlorophenyl)-4-cyclohexyl-1-butanone. The suspension turnes at 82° C. under stirring into a dark solution which is stirred at 100-105° C. inner temperature for 5.5 hours. To the reaction mixture 30 ml of ethanol is added, at 38-40° C. the crystallization starts. The reaction mixture is cooled under stirring to room temperature and stirred for 30 minutes. The crystals are filtered off in vacuum, washed with ethanol to obtain 5.85 g white crystals of the title compound, mp: 170-171° C.

### 26.) Preparation of 2-[[[4-(2,5-dimethoxyphenyl)-5-cyclohexylmethylthiazol-2yl]amino]carbonyl]-5,7-dimethyl-1H-indolee

N-(aminothioxomethyl)-5,7-dimethyl-1H-indole-2-carboxamide and 2-bromo-1-(2,5-dimethoxyphenyl)-2-cyclohexyl-1-ethanone are reacted according to Example 25. The crude product is purified by chomatography using toluene-methanol 10:1 mixture eluent to obtain the title compound as white crystals, mp: 203-205° C.

### 27.) Preparation of 2-[[[4-(2,5-dimethoxy-4-chlorophenyl)-5-cyclohexylethyl-thiazol-2-yl]amino]carbonyl]-3,5-dimethyl-1 H-indole-1-acetic acid methyl ester

N-(aminothioxomethyl)-3,5-dimethyl-1-(methoxycarbonylmethyl)-1H-indole-2-carboxamide and 2-bromo-1-(2,5-dimethoxy-4chlorophenyl)4-cyclohexyl-1-butanone are reacted according to Example 25. White crystals of the title compound are obtained, mp: 166-168° C.

### 28.) Preparation of the mixture of 2-[[[4-(2,5-dimethoxy-4-chlorophenyl)-5-cyclohexylethylthiazol-2-yl]amino]carbonyl]-4,5-dimethyl-1H-indole-1-acetic acid methyl ester and 2-[[[4-(2,5-dimethoxy-4-chlorophenyl)-5-cyclohexylethylthiazol-2-yl]amino]carbonyl]-5,6-dimethyl-1H-indole-1-acetic acid methyl ester

The mixture of N-(aminothioxomethyl)-4,5-dimethyl-1-(methoxycarbonylmethyl)-1H-indole-2-carboxamide and N-(aminothioxomethyl)-5,6-dimethyl-1-(menthoxycarbonyl-methyl)-1H-indole-2-carboxamide is reacted with 2-bromo-1-(2,5-dimethoxy-4-chloro-phenyl)-4-cyclohexyl-1-butanone according to Example 25. White crystals of the title compound are obtained, mp: 147-148° C.

### 29.) Preparation of 2-[[[4-(2,5-dimethoxy-4-chlorophenyl)-5-cyclohexylethyl-thiazol-2yl]amino]carbonyl]-1H-indole-1-propionic acid ethyl ester

N-(aminothioxomethyl)-1-(ethoxycarbonylethyl)-1H-indole-2-carboxamide and 2-bromo-1-(2,5-dimethoxy-4-chlorophenyl)-4-cyclohexyl-1-butanone are reacted according to Example 25. Butter-coloured crystals of the title compound are obtained, mp: 79-85° C.

### 30.) Preparation of 2-[[[4-(2,5-dimethoxy-4-chlorophenyl)-5-octylthiazol-2-yl]amino]carbonyl]-1H-indole-1-propionic acid ethyl ester

N-(aminothioxomethyl)-1-(ethoxycarbonylethyl)-1H-indole-2-carboxamide and 2-bromo-1-(2,5-dimethoxy-4-chlorophenyl)-1-decanone are reacted according to Example 25. Yellow-coloured crystals of the title compound are obtained, mp: 59-61° C.

### 31.) Preparation of 2-[[[4-(5-bromo-2,4-dimethoxyphenyl)-5-cyclohexylethyl-thiazol-2yl]amino]carbonyl]-1H-indole-1-acetic acid methyl ester

N-(aminothioxomethyl)-1-(methoxycarbonylmethyl)-5,7-dimethyl-1H-indole-2-carboxamide and 2-bromo-1-(5-bromo-2,4-dimethoxyphenyl)-4-cyclohexyl-1-butanone are reacted according to Example 25. Yellow crystals of the title compound are obtained, mp: 110-114° C.

### 32.) Preparation of 3-[[2-[[[4-(2,5-dimethoxy-4-methylphenyl)-5-cyclohexylethyl-thiazol-2-yl]amino]carbonyl]-5,7-dimethyl-1H-indole-1-yl]methyl]benzoic acid methyl ester

N-(aminothioxomethyl)-5,7-dimethyl-1-(3-methoxycarbonylbenzyl)-1H-indole-2carboxamide and 2-bromo-1-(2,5-dimethoxy-4-methylphenyl)-4-cyclohexyl-1-butanone are reacted according to Example 25. Yellow crystals of the title compound are obtained, mp: 219-220° C.

### 33.) Preparation of 3-[[2-[[[4-(5-bromo-2,4-dimethoxyphenyl)-5cyclohexyl-ethylthiazol-2-yl]-amino]carbonyl]-5,7-dimethyl-1H-indole-1-yl]-methyl]benzoic acid methyl ester

N-(aminothioxomethyl)-5,7-dimethyl-1-(3-methoxycarbonylbenzyl)-1H-indole-2-carboxamide and 2-bromo-1-(5-bromo-2,4-dimethoxyphenyl)-4-cyclohexyl-1-butanone are reacted according to Example 25. White crystals of the title compound are obtained, mp: 218-220° C.

### 34.) Preparation of 3-[[2-[[[4-(2,5-dimethoxy-4-chlorophenyl)-5-octylthiazol-2-yl]amino]carbonyl]-2,5-dimethyl-1H-indole-1-yl]methyl]benzoic acid methyl ester

N-(aminothioxomethyl)-2,5-dimethyl-1-(3-methoxycarbonylbenzyl)-1H-indole-2-carboxamide and 2-bromo-1-(2,5-dimethoxy-4-chlorophenyl)-1-decanone are reacted according to Example 25. White crystals of the title compound are obtained, mp: 165-166° C.

### 35.) Preparation of the mixture of 2-[[[4-(2,5-dimethoxy-4-methylphenyl)-5-cyclohexylethylthiazol-2-yl]amino]carbonyl]-4,5-dimethyl-1H-indole-1-acetic acid methyl ester and 2-[[[4-(2,5-dimethoxy-4-methylphenyl)-5-cyclohexylethylthiazol-2-yl]amino]carbonyl]-5,6-dimethyl-1H-indole-1-acetic acid methyl ester

The mixture of N-(aminothioxomethyl)-4,5-dimethyl-1-(methoxycarbonylmethyl)-1H-indole-2-carboxamide and N-(aminothioxomethyl)-5,6-dimethyl-1-(methoxycarbonyl-methyl)-1H-indole-2-carboxamide is reacted with 2-bromo-1-(2,5-dimethoxy-4-methylhenyl)-4-cyclohexyl-1-butanone according to Example 25. White crystals of the title compound are obtained, mp: 112-125° C.

### 36.) Preparation of 2-[[[4-(2,5-dimethoxy-4-chlorophenyl)-5-cyclohexylmethyl-thiazol-2-yl]amino]carbonyl]-5,7-dimethyl-1H-indole-1-acetic acid methyl ester

The solution of 1.8 g (4 mmol) of 1-(2,5-dimethoxy-4-chlorophenyl)-2-bromo-2-cyclohexylmethylthio-1-ethanone and 10 ml N,N-dimethylformamide is added to the suspension of 0.65 g (2 mmol) of N-(aminothioxomethyl)-5,7-dimethyl-1-(methoxycarbonylmethyl)-1H-indole-2-carboxamide and 5 ml of N,N-dimethylformamide and the reaction mixture is stirred at 95° C. for 3 hours. The dark-yellow solution is cooled down and 30 ml of water is added. From the precipitated amorphous material the solvent is decanted, 25 ml of ethanol is added to crystallize the product, which is filtered off and dried at room temperature. 0.9 g yellow crystalline product is obtained, mp: 143-145° C.

### 37.) Preparation of 2-[[[4-(2,5-dimethoxy-4-chlorophenyl)-5-cyclohexylthio-methylthiazol-2-yl]amino]carbonyl]-5,7-dimethyl-1H-indole-1-acetic acid methyl ester

1-(4-chloro-2,5-dimethoxyphenyl)-2-bromo-2-cyclohexylthiomethyl-1-ethanone and N-(aminothioxomethyl)-5,7-dimethyl-1-(methoxycarbonylmethyl)-1H-indole-2-carboxamide are reacted in N,N-dimethylformamide as described above. Mp: 130-136° C.

### 38.) Preparation of 2-[[[4-(2,5-dimethoxy-4-chlorophenyl)-5-cyclohexyl-ethylthiazol-2-yl]amino]carbonyl]-5,7-dimethyl-1H-indole-1-acetic acid

59.28 g (1.482 mol) of sodium hydroxide is dissolved in 3500 ml ethanol and to the solution is added under stirring 308.27 g (0.494 mol) of 2-[[[4-(4-chloro-2,5-dimethoxyphenyl)-5-cyclohexylethylthiazol-2-yl]amino]carbonyl]-5,7-dimethyl-1H-indole-1-acetic acid methyl ester. The mixture is stirred for 2 hours at 50-55° C. inner temperature, the opaque solution is filtered, then acidified at that temperature with 510 ml (534 g=8.892 mol) of acetic acid. The thick suspension is cooled to room temperature, the crystals are filtered off in vacuum, thoroughly washed with ethanol. 291.66 g white crystals of the title compound are obtained, mp: 227-228° C.

### 39.) Preparation of 2-[[[4-(2,5-dimethoxy-4-chlorophenyl)-5-cyclohexylethyl-thiazol-2-yl]amino]carbonyl]-3,5-dimethyl-1H-indole-1-acetic acid

From 2-[[[4-(2,5-dimethoxy-4-chlorophenyl)-5-cyclohexylethylthiazol-2-yl]amino]carbonyl]-3,5-dimethyl-1H-indole-1-acetic acid methyl ester according to Example 38., using hydrochloric acid of 10% for acidification, white crystals of the title compound are obtained, mp: 151-160° C.

### 40.) Preparation of 2-[[[4-(2,5-dimethoxy-4-chlorophenyl)-5-cyclohexylethylthiazol-2-yl]amino]carbonyl]-5-methyl-1H-indole-1-acetic acid

From 2-[[[4-(2,5-dimethoxy-4-chlorophenyl)-5-cyclohexylethylthiazol-2-yl]amino]carbonyl]-5-methyl-1H-indole-1-acetic acid methyl ester according to Example 38., using 1:1 hydrochloric acid solution for acidification, white crystals of the title compound are obtained, mp: 204-208° C.

### 41.) Preparation of the mixture of 2-[[[4-(2,5-dimethoxy-4-chlorophenyl)-5-cyclohexylethylthiazol-2-yl]amino]carbonyl]-4,5-dimethyl and -5,6-dimethyl-1H-indole-1-acetic acid

From the mixture of 2-[[[4-(2,5-dimethoxy-4-chlorophenyl)-5-cyclohexylethyl-thiazol-2-yl]amino]carbonyl]-4,5-dimethyl and -5,6-dimethyl-1H-indole-1-acetic acid methyl ester according to Example 38., using hydrochloric acid of 10% for acidification, white crystals of the title compound are obtained, mp: 198-200° C.

### 42.) Preparation of 2-[[[4-(2,5-dimethoxy-4-methylphenyl)-5-cyclohexylethyl-thiazol-2-yl]amino]carbonyl]-3,5-dimethyl-1H-indole-1-acetic acid

From 2-[[[4-(2,5-dimethoxy-4-methylphenyl)-5-cyclohexylethylthiazol-2-yl]amino]carbonyl]-3,5-dimethyl-1H-indole-1-acetic acid methyl ester according to Example 38., white crystals of the title compound are obtained, mp: 150-152° C.

### 43.) Preparation of 2-[[[4-(2,5-dimethoxy-4-methylphenyl)-5-cyclohexylethyl-thiazol-2-yl]amino]carbonyl]-5-methoxy-1H-indole-1-acetic acid

From 2-[[[4-(2,5-dimethoxy-4-methylphenyl)-5-cyclohexylethylthiazol-2-yl]amino]carbonyl]-5-methoxy-1H-indole-1-acetic acid methyl ester according to Example 38., white crystals of the title compound are obtained, mp: 163-165° C.

### 44.) Preparation of 2-[[[4-(5-bromo-2,4-dimethoxyphenyl)-5-cyclohexylethyl-thiazol-2-yl]amino]carbonyl]-5,7-dimethyl-1H-indole-1-acetic acid

From 2-[[[4-(5-bromo-2,4-dimethoxyphenyl)-5-cyclohexylethylthiazol-2-yl]amino]carbonyl]-5,7-dimethyl-1H-indole-1-acetic acid methyl ester according to Example 38., white crystals of the title compound are obtained, mp: 221-222° C.

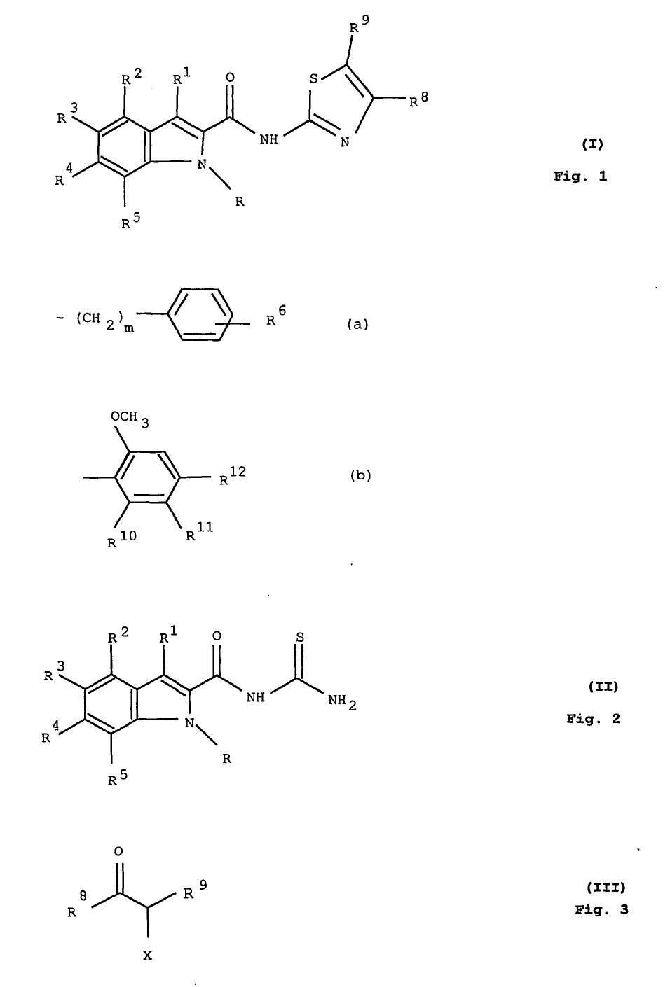

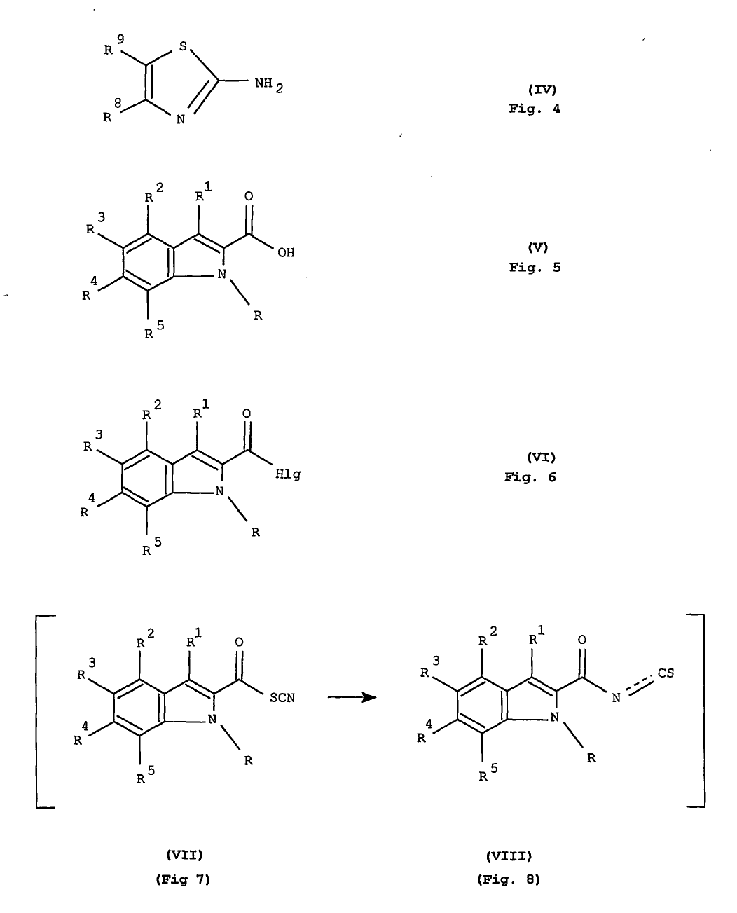

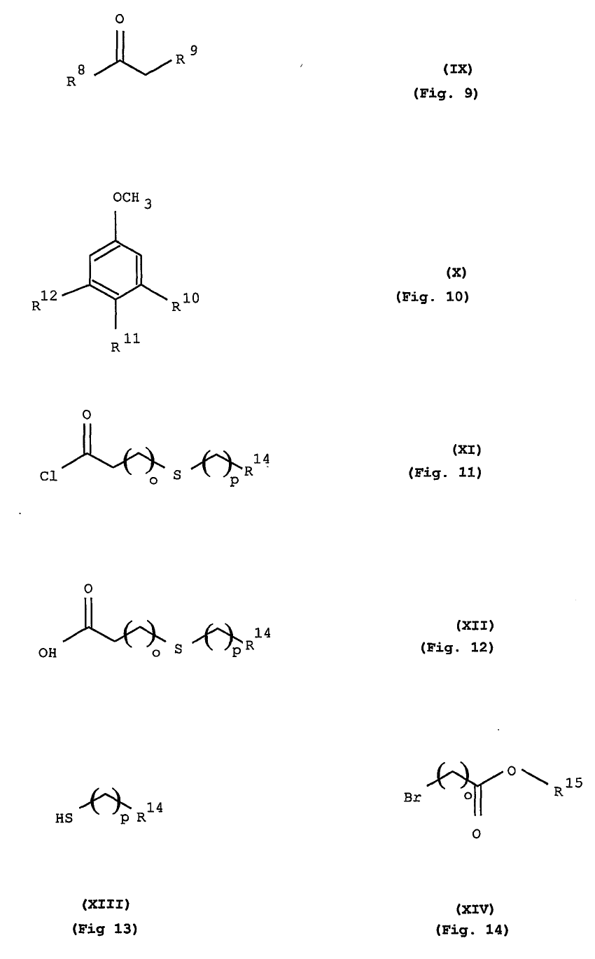

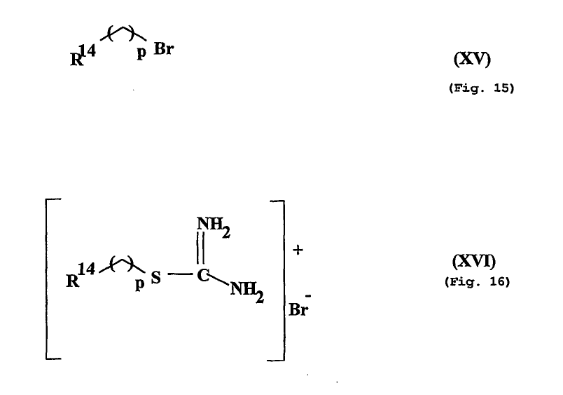

FIG. 1 shows the general formula (I), FIG. 2 shows the general formula (II), FIG. 3 shows the general formula (III), FIG. 4 shows the general formula (IV), FIG. 5 shows the general formula (V), FIG. 6 shows the general formula (VI), FIG. 7 shows the general formula (VII), FIG. 8 shows the general formula (VIII), FIG. 9 shows the general formula (IX), FIG. 10 shows the general formula (X), FIG. 11 shows the general formula (XI), FIG. 12 shows the general formula (XII), FIG. 13 shows the general formula (XIII), FIG. 14 shows the general formula (XIV), FIG. 15 shows the general formula (XV) and FIG. 16 shows the general formula (XVI).

1.) Process for the preparation of a compound of the general formula (I), pharmaceutically acceptable salts and solvates thereof-wherein
R¹ stands for hydrogen or methyl group,
R², R³, R⁴, R⁵ stand independently from each other for hydrogen, methyl, ethyl, hydroxyl, acetyloxy, methoxy, ethoxy, methyltio, trifluoromethyl or amino group or halogen atom,
R stands for hydrogen, a —(CH₂)ₙR⁶ group or a group of the general formula a.),
wherein
R⁶ stands for carboxyl or a —COOR⁷ group,
R⁷ stands for a C₁₋₄ alkyl group,
n=1, 2, 3, 4 or 5,
m=zero or 1,
R⁸ stands for a substituted phenyl group of the general formula b.),
wherein
R¹⁰ stands for hydrogen or methoxy group,
R¹¹ stands for hydrogen, methyl, ethyl, isopropyl, methoxy or ethoxy group or halogen atom,
R¹² stands for hydrogen, methyl, ethyl or methoxy group or halogen atom, or
R¹¹ and R¹² form together a methylenedioxy group,
R⁹ stands for a —CH₂—R¹³, —(CH₂)₂—R¹³ —S—CH₂—R¹³, —CH₂—S—R¹³ or C₅₋₈ alkyl group,
wherein
R¹³ stands for C₅₋₇ cycloalkyl group, with the proviso that R¹⁰, R¹¹ and R¹² can not stand at the same time for hydrogen-,
which comprises reacting an N-(amino-thioxo-methyl)-1H-indole-2-carboxamide of the general formula (II), wherein R¹, R², R³, R⁴, R⁵ and R are as defined above, with an α-halogen-ketone of the general formula (III), wherein X stands for halogen atom, R⁸ and R⁹ are as defined above and transforming the compound of the general formula (I) or its solvate thus obtained into its salt or liberating from its salt.

2.) Process according to claim 1, which comprises carrying out the reaction in the presence of a solvent.

3.) Process according to claim 2 which comprises using as solvent an aprotic solvents.

4.) Process according to claim 1, which comprises carrying out the reaction between room temperature and 120° C.

5.) Compounds of the general formula (II), wherein
R¹ stands for hydrogen or methyl group
R², R³, R⁴, R⁵ stand independently from each other for hydrogen, methyl, ethyl, hydroxyl, acetyloxy, methoxy, ethoxy, methyltio, trifluoromethyl or amino group or halogen atom,
R stands for hydrogen, a —(CH₂)ₙ, R⁶ group or a group of the general formula a.),
wherein
R⁶ stands for carboxyl or a —COOR⁷ group
R⁷ stands for a C₁₋₄ alkyl group
n=1,2,3,4 or 5
m=zero or 1-.

6.) Process for the preparation of a compound of the general formula (II), wherein
R¹ stands for hydrogen or methyl group,
R², R³, R⁴, R⁵ stand independently from each other for hydrogen, methyl, ethyl, hydroxyl, acetyloxy, methoxy, ethoxy, methyltio, trifluoromethyl or amino group or halogen atom,
R stands for hydrogen, a —(CH₂)ₙ R⁶ group or a group of the general formula a.),
wherein
R⁶ stands for carboxyl or a —COOR⁷ group,
R⁷ stands for a C₁₋₄ alkyl group,
n=1,2,3,4 or 5,
m=zero or 1-,
which comprises transforming an 1H-indole-2-carboxylic acid of the general formula (V), wherein R¹, R², R³, R⁴, R⁵ and R are as defined above, into an 1H-indole-2-carboxylic acid halogenide of the general formula (VI), wherein Hlg stands for halogen and R^{1,}R², R³, R⁴, R⁵ and R are as defined above, reacting the compound of the general formula (VI), thus obtained with potassium-thiocyanate and reacting the isothiocyanate of the general formula (VIII), thus obtained, wherein R¹, R², R³, R⁴, R⁵ and R are as defined above, with ammonia or ammonium hydroxide.

7.) Process according to claim 6, which comprises carrying out the reaction of the compound of the general formula (VI) with potassium-thiocyanate in the presence of a diprotic-aprotic solvent.

8.) Compounds of the general formula (III), wherein R⁸ stands for a substituted phenyl group of the general formula b.),
wherein
R¹⁰ stands for hydrogen or methoxy group,
R¹¹ stands for hydrogen, methyl, ethyl, isopropyl, methoxy or ethoxy group or halogen atom,
R¹² stands for hydrogen, methyl, ethyl or methoxy group or halogen atom, or
R¹¹ and R¹² form together a methylenedioxy group,
R⁹ stands for an —S—CH₂—R¹³, or —CH₂—S—R¹³ group,
wherein
R¹³ stands for C₅₋₇ cycloalkyl group, with the proviso that R¹⁰, R¹¹ and R¹² can not stand at the same time for hydrogen.

9.) Process for the preparation of a compound of the general formula (III), wherein R⁸ stands for a substituted phenyl group of the general formula b.),
wherein
R¹⁰ stands for hydrogen or methoxy group,
R¹¹ stands for hydrogen, methyl, ethyl, isopropyl, methoxy or ethoxy group or halogen atom,
R¹² stands for hydrogen, methyl, ethyl or methoxy group or halogen atom, or
R¹¹ and R¹² form together a methylenedioxy group,
R⁹ stands for an —S—CH₂—R¹³, or —CH₂—S—R¹³ group,
wherein
R¹³ stands for C₅₋₇ cycloalkyl group, with the proviso that R¹⁰, R¹¹ and R¹² can not stand at the same time for hydrogen-,
which comprises acylating a methoxy-benzene of the general formula (X), wherein R¹⁰, R¹¹, and R¹² are as defined above, with an acid-choride of the general formula (XI), wherein R¹⁴ stands for a C₅₋₇ cycloalkyl group, o stands for 1 or 2, p stands for zero or 1, in the presence of a Lewis acid, and halogenating the compound of the general formula (IX), wherein R⁹ stands for —S—CH₂—R¹³ or —CH₂—S—R¹³

10.) Process according to claim 9, which comprises using titanium-tetrachloride or aluminium-chloride as Lewis acid.

11.) Process according to claim 3 wherein the aprotic solvent is N,N dimethylformamide or N-methyl-2-pyrrolidone.

12.) Process according to claim 4 which comprises carrying out the rection between 80° C. and 120° C.

13.) Process according to claim 7 wherein the diprotic-aprotic solvent is acetone or methylethyl-ketone.
